(19) United States
(12) Patent Application Publication
(10) Pub. No.: US 2003/0098271 A1
(43) Pub. Date: May 29, 2003

(54) Capsule and tray systems for combined sample collection, archiving, purification, and PCR

(76) Inventors: Ralph Somack, Oakland, CA (US); Gary Lim, San Francisco, CA (US)

Correspondence Address: KILYK & BOWERSOX, P.L.L.C., 3603-E Chain Bridge Road, Fairfax, VA, 22030, US

(21) Appl. No.: 09/994,495

(22) Filed: Nov. 26, 2001

(51) Int. Cl. C12Q 1/68; C02F 1/44

(52) U.S. Cl. 210/295; 435/6

(57) ABSTRACT

The present invention relates to a biological sample collection, archiving, purification, and manipulating system and methods for collecting, archiving, purifying, and manipulating biological samples. The system can include a plurality of devices, each having a species-immobilizing filter within a tubular body. The body includes first and second end openings and one or more removable caps or sealing devices for sealing the end openings. A plurality of the devices can be positioned in an array tray for simultaneously archiving, purifying, or reacting respective samples collected in the respective devices. In another embodiment, a plate system is provided that includes a plate having a plurality of through-holes with each through-hole having a species-immobilizing filter disposed therein. The plate system also includes sealing devices such as sealing trays or removable caps for sealing first and second end openings of each through-hole of the plate.

### FIELD OF THE INVENTION

The present invention relates to a sample collection and purification system for biological samples. The present invention also relates to a method for collecting and purifying biological samples.

### BACKGROUND OF THE INVENTION

Many systems, devices, methods, and processes have been developed for the collection and purification of biological samples. To fully analyze the contents of such collected samples, it is often necessary to purify, archive, and analyze the samples. Such methods include transferring a sample from a collection tray to a purification tray, eluting the sample into an archive tray, and transferring the sample to an analysis tray. Frequently, polymerase chain reaction (hereafter “PCR”) is subsequently utilized to amplify nucleic acid components purified from such samples. With each of these process steps comes the potential for cross-contamination between sample collection wells when a multi-well device is used. Other potential problems encountered include cumulative variability introduced during the multiple sample transfer steps, loss or dilution of sample through sample clinging or evaporation, introduction of contaminants, and/or sample misidentification.

Sample collection systems that are known include those described in European Patent Application No. 0 331 127; and in U.S. Pat. Nos. 5,910,246; 4,642,220; 5,665,247; 6,277,648 B1; 6,270,980 B1; 6,153,425; 6,043,080; 5,853,586; 5,955,351; 5,955,271; 5,833,927; 5,783,087; 5,552,325; 5,436,129; 5,356,596; 5,124,041; 5,043,082; 4,990,442; and Re. 35,306. Other known systems include those described in Japanese Patent Publications JP 10-257887, and JP 10-136999.

All of these patents, patent applications, and publications, and all others mentioned herein, are incorporated in their entireties herein, by reference.

### SUMMARY OF THE INVENTION

The present invention provides a system including a plurality of devices, in each of which a biological sample can be collected, archived, purified, and/or subjected to PCR, transcription, reverse transcription (RT), reverse transcription PCR (RTPCR), or other reaction, without the need to transfer the sample to one or more additional systems or devices. The present invention also provides a system whereby a plurality of such devices can be organized so that respective samples collected in the respective devices can simultaneously be archived, purified, and subsequently analyzed, for example, subjected to PCR, transcription, RT, RTPCR, or another reaction.

According to an embodiment of the present invention, a purification tray system for processing a plurality of fluid samples is provided. This system comprises a plurality of biological sample purification devices. Each device comprises a tubular body having a first end with a first end opening, and a second end with a second end opening. A species-immobilizing filter is secured within the tubular body to collect a target analyte, for example, a nucleic acid molecule or a nucleic acid molecule fragment. The species-immobilizing filter can collect the target analyte through a size-exclusion interaction, a binding interaction, an affinity interaction, or through other filtering mechanisms known to those skilled in the art. Each device further includes a removable cap adapted to seal either the first end opening or the second end opening, and can include two removable caps adapted to respectively seal the first end opening and the second end opening of the device. The purification tray system also includes a tray having a surface adapted to individually seal each of the first end openings or the second end openings of two or more of the plurality of devices.

According to another embodiment of the invention, a combined multiple device and array tray system is provided whereby PCR, RT, RTPCR, or another reaction can be effectively carried out on a plurality of target nucleic acid or nucleic acid fragment samples simultaneously. The target analyte can be bound to or trapped in or on the species-immobilizing filter within each device. Subsequently, the multiple devices arranged in the array tray are subject to conditions that enable PCR amplification.

According to yet another embodiment of the present invention, a sample collection, archiving, purification, and reaction device is provided in the form of a plate system. The plate system includes a plate having a plurality of through-holes with each through-hole having a species-immobilizing filter secured therein. The plate system also includes sealing trays or removable caps for sealing first and second end openings or each through-hole of the plate. A plurality of respective samples can be collected in the respective through-holes of the plate, archived, purified, analyzed, and/or subjected to PCR, transcription, RT, RTPCR, or another manipulation.

Different types of devices can be designed for different specific applications, such as RNA, DNA, or total nucleic acid purification from blood samples, plant or animal cell samples, tissue samples, or microorganism samples. The devices of the present invention can be bar-coded for identifying the type of membrane, type of pre-loaded agent, intended application, and/or for sample identification.

The present invention is further directed to devices that combine the features of sample collection trays, nucleic acid purification trays, archiving trays and PCR and other reaction trays, into a single, universal tray that can be processed on a work station. For example, the multi-purpose system of the present invention can provide a tray capable of automation in robotic work stations such as the Applied Biosystems automated Model 6700 work station. Alternatively, the multi-purpose system of the present invention can provide a tray capable of use in manually operated workstations such as the Applied Biosystems Model 6100 purification work station.

According to a method of the present invention, a biological sample purification device is provided that includes a tubular body having a first end with a first end opening, and a second end with a second end opening, and a species-immobilizing filter secured within the tubular body. The method involves introducing a biological sample into the tubular body through at least one of the first end opening and the second end opening. By causing a pressure differential, for example, through gravity, capillary action, vacuum, or pressurized fluid, the biological sample is moved across the species-immobilizing filter such that a target analyte within the biological sample is immobilized on the species-immobilizing filter. After the species-immobilization step, an additional purification step, and/or a washing step, can be performed to further isolate the target analyte on the filter. Optionally, one or more agents, reagents, or other components can be added prior to sealing. Subsequently, the first end opening and second end opening of the device are sealed, with either removable caps, sealing trays, or a combination thereof, to form a sealed device. The sealed device can subsequently be analyzed or subjected to PCR, transcription, RT, RTPCR, or another reaction to produce a product of the target analyte. If subjected to PCR, transcription, RT, or RTPCR, the product in the sealed device can subsequently be analyzed.

The sealed device, whether containing crude sample or purified sample, and whether or not subjected to a reaction such as PCR, transcription, RT, or RTPCR, can be archived for an extended period of time such as 100 hours or more, and protects the sample sealed therein from evaporation, contamination, and leaking.

The present invention is especially well suited for collecting, archiving, purifying, PCR, transcription, RT, or RTPCR processing, and analyzing samples such as blood samples and other nucleic acid-containing samples.

In another embodiment of the invention a method is provided for the collection of biological samples in the sealable devices disclosed herein. In another embodiment, the present invention provides a method for purifying a biological sample collected in the device. In yet another embodiment of the invention, a method is provided for archiving biological samples.

The embodiments of the present invention provide several advantages over prior sample collection, archiving, purification, and PCR systems. The present invention eliminates the need to transfer samples from a collection vessel to a purification tray. The present invention also reduces the potential for sample mis-identification associated with transfer steps. Another advantage of the present invention is the reduction in purification time by the elimination of post-purification sample elution, dilution, and reaction tray transfer steps. The present invention also reduces the potential for cross-contamination among multiple sample containment devices. In addition, the present invention eliminates the need to use additional reagents, such as sample elution and dilution solutions, and the need to load such additional reagents into collection, purification, and reaction instruments, assemblies, or devices.

### BRIEF DESCRIPTION OF THE DRAWINGS

The present invention will become more fully understood from the detailed description given herein and the accompanying drawings. The accompanying drawings and detailed description of the present invention set forth herein are illustrative only and are not intended to limit the scope of the present invention defined by the appended claims. In the accompanying drawings:

FIG. 1 is a perspective, exploded, side view in partial phantom of a capsular device including removable and sealable end caps, according to an embodiment of the present invention;

FIG. 2 is a perspective view of a system according to an embodiment of the present invention including an array tray having through-holes and a plurality of capsular devices each partially inserted in a respective through-hole of the array tray;

FIG. 3 is a perspective view of the system shown in FIG. 2 wherein the plurality of capsular devices are fully inserted in the through-holes of the array tray and the removable caps remain on both ends of each capsular device;

FIG. 4 is a perspective view of the system of FIG. 3 wherein the caps at the lower ends (as shown) of the capsular devices have been removed;

FIG. 5 is an exploded perspective view of a system according to an embodiment of the present invention including an array tray having through-holes, a plurality of capsular devices each partially inserted in a respective through-hole of the array tray, and a sealing tray having a plurality of recesses for receiving the lower ends (as shown) of the plurality of capsular devices;

FIG. 6 is a perspective view of the system shown in FIG. 5 in an assembled state wherein the lower ends of the plurality of capsular devices are received in the recesses of the sealing tray;

FIG. 7 is a perspective, partial cut-away view of yet another embodiment of the present invention wherein a plate system is provided having a plurality of through-holes and a species-immobilizing filter disposed in each through-hole;

FIG. 8 is a cross-sectional view taken through line VIII-VIII of FIG. 7;

FIG. 9 is a perspective exploded view of an assembly including the plate system of FIG. 7 disposed above a waste collection tray;

FIG. 10 is a cross-sectional view of the waste collection tray shown in FIG. 9 taken through line X-X of FIG. 9; and

FIG. 11 is a cross sectional view of the plate system shown in FIG. 7 having a sealing top plate and a sealing bottom plate adhered to the respective top and bottom surfaces of the system.

It is to be understood that both the foregoing general description and the following detailed description are exemplary and explanatory only and are intended to provide further explanation of the present invention, as claimed.

### DETAILED DESCRIPTION OF THE PRESENT INVENTION

According to an embodiment of the present invention, the invention relates to a system including an array tray and one or more individual biological sample containment devices, each device having a substantially cylindrical body, for example, with a tapered section, that snaps into a hole or recess in an array tray. Each device of the system is adapted for sample collection, archiving, purification, and carrying out a reaction of the sample such as a nucleotide polymerization reaction, PCR, transcription, RT, or RTPCR. Each device can incorporate a nucleic acid purification membrane that can be positioned in the body, for example, at or near the bottom of the body. Each device can also have removable caps at one or both ends of the body to protect the membrane and preserve the sample. The array tray can have a plurality of holes or recesses to receive a plurality of such devices.

According to an embodiment of the present invention, the system includes a sealing device such as a tray, plate, membrane, tape, or the like having a surface adapted to individually seal each of the first end openings of a plurality of the devices. The sealing device surface can include a plurality of recesses therein, adapted to receive the first ends of the plurality of devices respectively in the recesses. The relationship between the first ends of the devices and the plurality of recesses is such that when the first ends are received in the recesses the first end openings of the devices are sealed.

Sealing with the sealing device can be accomplished by a frictional fit, a threaded engagement between the first ends and the recesses, with adhesive, with gasket material, by compression fit, by a snap-lock engagement, or by other sealing means know to those skilled in the art.

According to an embodiment of the present invention, each device can include a removable cap for the first end thereof, and a second removable cap for the second end thereof. The caps can be designed to seal the respective first end and second end of the device by any of the sealing mechanisms described above in connection with the sealing device. The caps can be adapted to seal the first and second end openings of the device with sufficient integrity to prevent evaporation of a liquid sample stored in the device and to maintain the seal while withstanding the thermocycling necessary for nucleotide manipulating reactions such as polymerase chain reaction.

According to an embodiment of the present invention, the species-immobilizing filter of each device is positioned within the tubular body closer to the second end of the tubular body than to the first end of the tubular body. The tubular body can have a smaller diameter second end compared to the diameter of the first end of the body and can be tapered along the length of the tubular body or along a length of a portion of the tubular body. The tubular body can be cylindrical, oval, elliptical, square, triangular, or of any other suitable cross-section. The tubular body can be made of plastic or glass, for example, and can be rigid and inflexible.

According to an embodiment of the present invention, the species-immobilizing filter is positioned within the tubular body closer to the second end opening than to the first end opening. According to a particular embodiment, the ratio of (1) the distance from the species-immobilizing filter to the first end of the tubular body, to (2) the distance from the species-immobilizing filter to the second end of the tubular body, is greater than or equal to about 4:1, for example, greater than or equal to about 10:1. According to an embodiment of the present invention, the species-immobilizing filter of one or more of the devices is positioned at or adjacent to the second end of the respective tubular body, for example, such that when the second end of the tubular body is placed in or on a liquid sample, the sample can be absorbed into or onto the species-immobilizing filter.

The species-immobilizing filter is held in place within the tubular body. Any suitable device, system, and/or method can be used to hold the filter in the tubular body. The filter can be held in place by an adhesive, by a compression fit, by a ridge or shoulder on an inside surface of the tubular body, a ledge, retaining ring, or a bead of material integrally formed with or attached to the inside surface of the tubular body, or the like. The filter can be securely and/or permanently held within the tubular body.

According to embodiments of the present invention, the first end openings of a plurality of devices and the second end openings of a plurality of devices can be sealed by any combination of sealing devices including removable sealing caps and suitable sealing membranes or trays. According to an embodiment of the present invention, the first end openings of the plurality of devices are sealed with a first sealing device, and the second end openings of the plurality of devices are sealed with a second, different sealing device. The sealing devices can include an adhesive surface or layer, for example, including a pressure sensitive adhesive, an optically-curable adhesive, an ultraviolet radiation-curable adhesive, or the like.

The species-immobilizing filter used in each device can be a nucleic acid purification membrane. The species-immobilizing filter can be designed for absorption, adsorption, or other mechanisms for retaining a sample thereon or therein. According to an embodiment of the present invention, the species-immobilizing filter includes a porous structure which absorbs a liquid sample.

Those skilled in the art will recognize that the choice of filter medium will depend on the intended use of the well. For example, the filter medium might serve as a physical entrapment filter, a size exclusion filter, or it could serve as a solid phase interacting with a species in the liquid phase. The filter can preferably immobilize species upon contact, such as through an immunological interaction, any type of affinity interaction, or any type of chemical interaction. Examples of suitable filters include, but are not limited to, those of nitrocellulose, regenerated cellulose, nylon, polysulfone, glass fiber, blown microfibers, and paper. Suitable filters are available from a variety of sources, e.g., Schleicher & Schuell, Inc. (Keene, N.H.) and Millipore Corp. (Bedford, Mass).

Additional examples of suitable filters include microfiber filters of ultra-pure quartz (SiO₂), e.g., as manufactured by Whatman, Inc. (Tewksbury, Mass.) and sold under the trademarks QM-A and QM-B. QM-A filters are about 0.45 mm thick and retain particles of about 0.6 μm. QM-B filters are of the same composition as QM-A, but are two times thicker and therefore provide a longer tortuous path to flow. In one embodiment, a quartz or glass filter element is fired (e.g., at about 400° C.) prior to placement in the tubular body.

In another embodiment the filter medium is a porous element that acts as a frit, serving to contain a column packing material, e.g., reversed-phase or size-exclusion packings, or silica packings.

In accordance with an embodiment of the present invention, a filter or purification membrane for separating and binding a target analyte such as a biomolecule, is provided.

In processes requiring the separation and analysis of biomolecules such as proteins, nucleic acids, lipids, and carbohydrates, it is often convenient to bind the biomolecules to a solid matrix at some point in the process flow. Such binding allows materials that might be troublesome in further analysis to be removed while retaining a biomolecule or biomolecules of interest. A variety of support matrices and attachment chemistries have been developed in the art, depending on the particular biomolecule to be separated. These materials include, for example, nitrocellulose, DEAE-cellulose, derivatized glass beads, derivatized nylon, parchment, macrocyclic polyethers, polyvinylbutyral resin, polyvinylalcoholcollagen, polyvinylidene difluoride, and other polymers. As a specific example, diisothiocyanate (DITC) derivitized glass beads have been used as well as DITC functionalized glass fiber sheets. More recently polyvinylidene difluoride (PVDF) membranes have been used in blotting and electroblotting, and have proved to be relatively more useful than glass supports. The membrane bound molecules can be readily visualized with a variety of compound, for example, by staining, such as with coomassie blue for proteins.

According to embodiments of the present invention each device is preloaded with a component useful in a method carried out in the device. The component can be any of a variety of agents, reagents, solutions, preservatives, or other compositions including, but not limited to, powdered, particulate, solid, slurry, or mixed composition. An example of a pre-loadable agent is a lysing agent useful in lysing cellular components of a sample to release a species, such as a nucleic acid molecule or nucleic acid fragment. In a subsequent flushing or washing step, cellular components from the lysing are released and/or washed through the species-immobilizing filter such that a target nucleic acid or nucleic acid fragment, preferably alone, is retained, bound, isolated, and/or otherwise immobilized on or in the species-immobilizing filter.

The device can be pre-loaded with liquid reagents, such as guanidinium salts, that lyse cellular components of a sample, stabilize nucleic acids released from such components, and/or inactivate pathogens present in the sample. The pre-loaded reagents may also include, for example, sodium citrate, EDTA, or heparin, to prevent clotting of blood samples or provide other treatment to the sample. Lysis agents, stabilization agents, or clot prevention agents can be incorporated into or onto the purification membrane itself, for example, in a dried or semi-dried form. Such agents can be added instead, or additionally, after introduction of a sample into the device.

Other components that can be pre-loaded into the tubular device prior to use include preservatives, diluents, buffers, and the like, known by those skilled in the art.

The above, and other components can alternatively, or additionally, be introduced into the tubular device along with, or after, introduction of a biological sample and/or one or more other components.

According to exemplary methods of the present invention, a whole blood sample can be absorbed by a species-immobilizing filter positioned at an end of the tubular device. A lysing agent can then be introduced into the tubular device under conditions sufficient to lyse whole blood cells from the sample to release and/or fragment nucleic acid molecules from the sample. Subsequently, a washing fluid can be introduced in the tubular device to wash away cellular components other than the target nucleic acid molecule or fragment. Passing lysing agents and washing solutions through the species-immobilizing filter can be effected by gravity or facilitated with the use of a vacuum source, pressure source, or centrifugal force applied to either end of the tubular device. In a subsequent operation, preservatives or other components can optionally be added to the device after which the device can be sealed and archived for extended periods of time, for example, for greater than one hundred (100) hours, for greater than one (1) week, for greater than three (3) months, and for greater than ten (10) years. In an alternative or subsequent operation, a reaction solution can be introduced into the tubular device, followed by sealing, and carrying out a reaction of a target analyte. The reaction solution can be a PCR solution, an RT solution, an RTPCR solution, and the like, depending upon the desired reaction to be carried out.

According to another embodiment of the present invention, a pre-purified target analyte is introduced into the tubular device followed by, or simultaneously with, the introduction of a reaction solution having sufficient components to affect the desired reaction of the target analyte.

The present systems and methods are also applicable to biological samples other than whole blood samples. Other exemplary samples that can be manipulated with the device of, and according to the methods of, the present invention include animal cell lysates, plant cell lysates, tissue extracts, and the like.

According to yet another embodiment of the present invention, any number of biological samples or tubular devices can be manipulated simultaneously. For example, a system comprising any number of tubular devices can be sealed by a sealing device that is designed to seal a number of tubular devices in excess of the number sealed by the system.

According to yet another embodiment of the present invention, the tubular devices and array tray are replaced with a plate system that includes a plate having a plurality of through-holes. In each of the through-holes, a species-immobilizing filter is secured. The plate systems include sealing trays or removable caps for sealing the end openings of each through-hole of the plate. The filter materials, configurations, dimensions, and additional components mentioned above with reference to other embodiments of the present invention would likewise be suitable in the plate system configuration of the present invention. The sealing device for the plate system can be an adhesive membrane, caps, plugs, tape, or the like.

Methods according to embodiments of the present invention include the introduction steps, sealing steps, and manipulating steps mentioned above and other useful process steps including the application of a pressure differential force on opposing sides of the species-immobilizing filter. The present invention is intended to cover these and other methods that would be obvious to those skilled in the art from the description of the systems of the present invention described above.

According to a method of the present invention, the target analyte, if present in the biological sample, is caused to be immobilized by the species-immobilizing filter, and components of the biological sample other than the target analyte are removed from the species-immobilizing filter. This may be achieved, for example, by washing, centrifugation, and/or drawing a vacuum through the species-immobilizing filter. Purification solutions can be washed through the filter under vacuum, either manually or robotically to thereby filter the target analyte from the biological sample.

Causing the target analyte to be immobilized by the species-immobilizing filter can be achieved by lysing biological samples such as whole blood cells, plant or animal cells, or tissue extracts to release the target analyte from the biological sample. The released target analyte then contacts the surface, interior, or matrix of the filter where the target analyte becomes immobilized, for example, by a chemical binding reaction. Cellular samples can be lysed with lysing agents or chemicals to free nucleic acids and nucleic acid fragments, so as to affect binding, of at least one target nucleic acid analyte or nucleic acid fragment analyte, to the species-immobilizing filter. Also useful herein are blood products, such as plasma. The tissue extracts useful herein can also contact cell lysates already bound to the species-immobilizing filter.

The system can then be transferred by either manual or robotic means onto a surface or platform optionally pre-loaded with an adhesive or heat-sealable membrane material or tray. The adhesive or heat-sealable membrane can be permanently bonded to the bottom of the plate using either pressure or heat adhesives, such as a heat-sealable membrane. This step creates a non-leakable seal and converts the purification system into a reaction plate for PCR, transcription, RT, RTPCR, or the like.

Reaction reagents, for example, PCR solution, can be added to the wells, either manually or robotically, and the system can then be sealed across the tops of the wells in the array with an adhesive cover, for example, a pressure sensitive adhesive cover or an optically-curable adhesive cover. Other adhesives can be used including those adhesives known to those skilled in the art. By “optically-curable adhesive cover” herein is meant any covering material that can be cured, anchored, attached, or sealed onto an end opening of a device or over a system of the present invention by means of exposure of the cover to radiation. In a preferred embodiment, but not as a limitation herein, the radiation useful for curing the optically-curable adhesive cover is ultraviolet radiation.

The sealed system can be transferred to a thermal cycler or sequence detection system, for example, for sample analysis.

The present invention provides a purification system for processing a plurality of fluid samples, wherein the system includes a tray having a first surface and a plurality of recesses in the first surface, and a plurality of biological sample purification devices. Each device of the plurality of devices is adapted to be received in a respective one of the recesses. Each device comprises a tubular body having a first end, a first end opening, a second end, a second end opening, and a species-immobilizing filter within and secured to the tubular body.

The capsule and tray systems of the present invention can, in an embodiment of the present invention, be designed to perform as complete systems in the following way. A removable end cap on the tubular body is removed and sample is introduced into the body either directly onto the membrane or into a pre-loaded reagent, if used. This can be achieved, for example, by contacting an end opening of a device according to the invention to a blood sample generated on a person's body, such as a fingertip as by pricking the fingertip. The blood drop is caused to enter the end opening of the device and be absorbed by the filter, for example, by capillary action. The cap, which in a particular embodiment is adapted to snap onto the open end of the device to form a tight seal therewith, is reapplied to the end opening of the device to seal the end of the device. The sealed device can then be archived, categorized, analyzed, purified, shipped to a laboratory for further analysis, or sent to a library or other remote location for later use or study. A “library” herein can mean, for example, a collection of samples from various individuals or from the same individual over a time period. Bar-codes can be externally applied to the sealed devices for tracking and recordation.

At the analysis site, the capsule bar-codes can be scanned and the capsules can be snapped into a special holding tray designed to accept multiple capsule units. Bar-codes on the holding tray corresponding to capsule positions can also be scanned. The end caps can be removed and the devices in an array can be sealed and loaded onto the deck-space of a nucleic acid purification device, such as the Applied Biosystem Inc. Model 6700 robotic nucleic acid purification workstation or the Model 6100 manually operable nucleic acid purification workstation. The array tray can also seal the devices or a separate sealing device can be used.

A workstation utilizing the capsule and tray systems of the present invention performs sample purification by filtering various agents through the capsule membrane, for example, under the force of gravity, vacuum, pressure, or centrifugation. The robotic or manual workstation can transfer the device-holding array tray assembly onto a base sealing tray located at another deck-space position. The device-holding array tray assembly can be snapped into a base sealing tray, thereby providing a sealing membrane to the bottom of the tray assembly.

Thus, in one embodiment, the present invention provides a purification apparatus positioned on a vacuum or centrifugation platform of a device for vacuum filtering or centrifugally separating at least one target analyte from other components of a sample disposed on the species-immobilizing filter.

The robotic workstation can add pre-programmed, target-specific reaction master mixes to the capsules (or wells) for carrying out a desired reaction such as PCR. The workstation (if automated), or an operator (if the workstation is manual), can then seal the tops of the capsules (or wells) with a sealing device, for example, a pressure sensitive adhesive cover, an optically curable adhesive blanket or cover, or sealing caps.

The tray assembly can then be transferred to a reaction system or detection system, for example, a PCR sequence detection system such as the Applied Biosystems Inc. Model 7700 or 7900 HT device for target analyte amplification and analysis.

According to an embodiment of the present invention, a nucleic acid molecule or fragment can be subjected to real-time PCR in the system of the present invention. A discussion of real-time PCR is set forth at <http://dna-9.int-med.uiowa.edu/realtime.htm> and is incorporated in its entirety herein by reference. Real-time PCR involves real-time monitoring of the concentration of a target nucleic acid sequence. In the web page mentioned, the monitoring is accomplished by measuring relative fluorescence of a TAQMAN® (Roche Molecular Systems, Inc., Somerville, N.J.) fluorescing probe using an excitation source and a CCD array as provided in the Applied Biosystems, Inc. Model 7700 Sequence Detection System.

Experiments conducted using apparatus and methods of the present invention have demonstrated the feasibility of performing PCR on nucleic acids directly from the species-immobilizing filter without first eluting the sample.

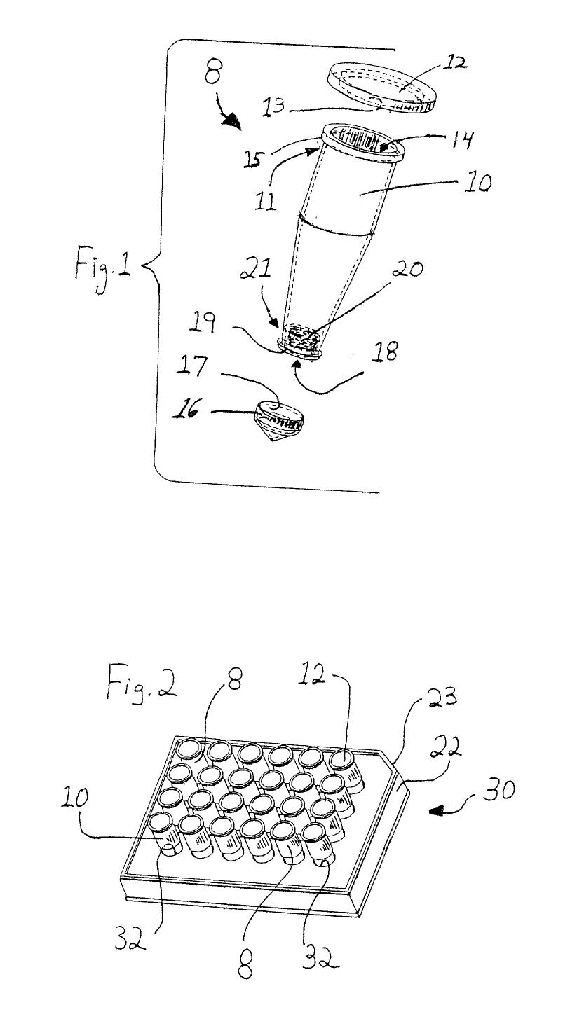

Referring now to the drawing figures, FIG. 1 is a perspective, exploded side view in partial phantom of a capsular device 8 useful in a system according to an embodiment of the present invention. The capsular device 8 includes a tubular body 10 having a first end 11 and a second end 21. At the first end 11 is a first end opening 14, and at the second end 21 is a second end opening 18. A removable sealing end cap 12 is provided for attaching to the first end 11 of the tubular body 10 and sealing the first end opening 14. A removable sealing end cap 16 is provided for attaching to the second end 21 of the tubular body 10 and sealing the second end opening 18. Although any suitable attachment mechanism or method can be used to attach the caps to the respective ends 11 and 21 of the tubular body 10, a snap-fit connection is shown. The snap fit connection is provided, for example, by designing the outer periphery of the first end 11 with a lip or rim having an outer surface 15, and including on a surface of cap 12 an inner lip or rim having an inner surface 13 wherein the inner surface 13 sealingly engages the outer surface 15 such that the cap 12 snaps onto the tubular body 10.

FIG. 1 also shows a filter 20 located at the lower end 21 of the tubular body 10, within the tubular body. The filter 20 is secured to the inner surface of the tubular body 10, by a compression fit, adhesive, flange, or other filter retaining devices known to those skilled in the art. In the embodiment depicted in FIG. 1, the filter defines a barrier between the two end openings of the tubular body, i.e., completely stretches across the inside diameter of the tubular body on a plane perpendicular to the longitudinal axis of the tubular body 10. Although circular end caps 12 and 16 are depicted, end caps of any suitable size and/or dimensions can be used.

FIG. 2 is a perspective view of a system 30 according to an embodiment of the present invention. The system 30 includes an array tray 22 having a plurality of through-holes 32 and a plurality of the capsular devices 8 shown in FIG. 1, with each device 8 being partially inserted in a respective through-hole 32 of the array tray 22. The array tray 22 can be of any suitable shape including, for example, square or rectilinear shapes. The array tray can include a squared-off corner or keyed region 23 to ensure proper orientation, registration, and/or alignment of the system in or on a workstation. The array tray 22 can contain the same number of through-holes as capsular devices. Trays containing 96 sample wells can be used as can array trays having larger and smaller numbers of samples.

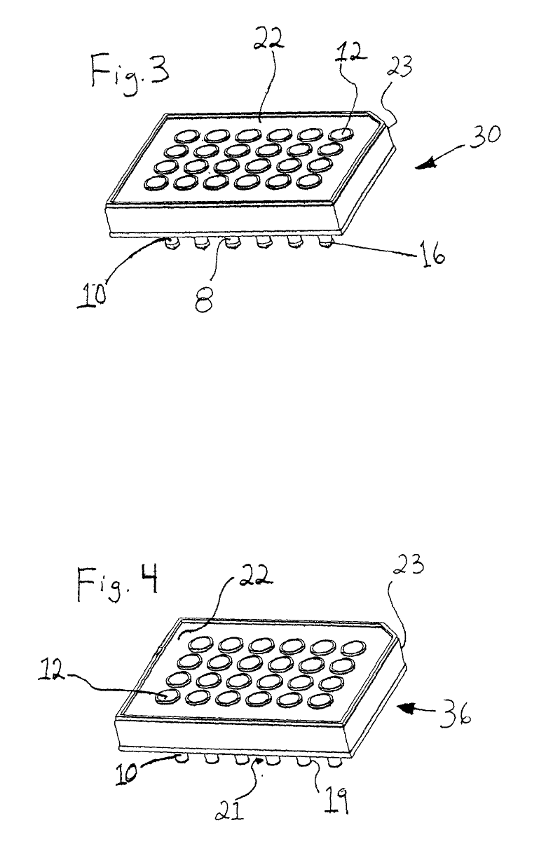

FIG. 3 is a perspective view of the system 30 shown in FIG. 2 wherein the plurality of capsular devices 8 are fully inserted in the through-holes of the array tray 22 and the removable caps 12 and 16 remain on the ends of each tubular body 10. The relation between the shape of capsular devices 8 and the through-holes 32 can be such that the devices 8 snap into the array tray 22.

FIG. 4 is a perspective view of a system 36 according to the present invention that is similar to the system 30 of FIG. 3 but wherein the removable caps 16 at the second ends 21 of the capsular devices 8 have been removed.

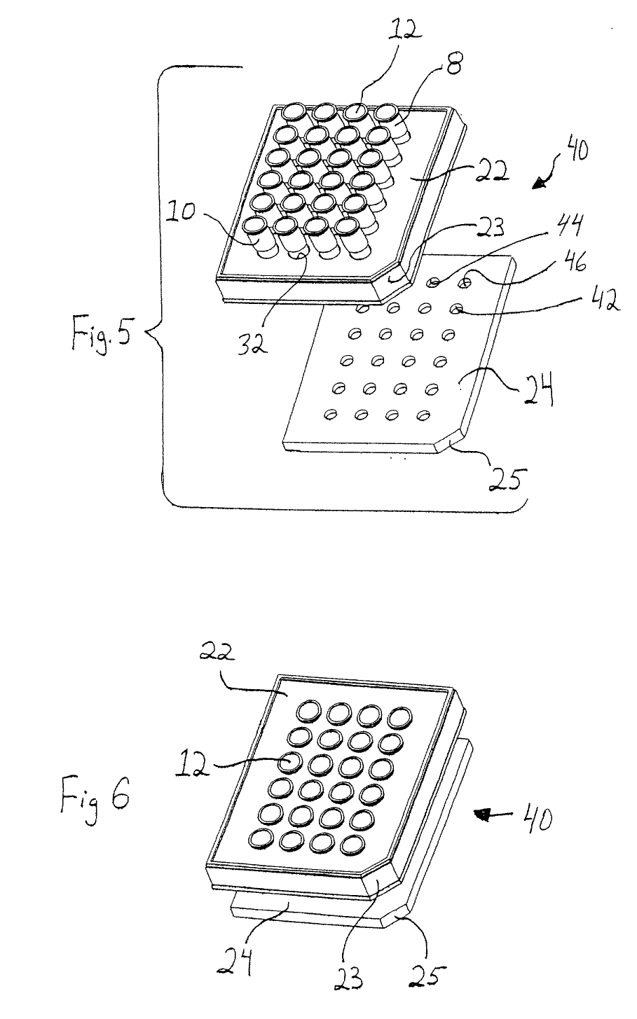

FIG. 5 is an exploded perspective view of a system 40 according to an embodiment of the present invention that is similar to the system 36 shown in FIG. 4 but which further includes as a sealing device a sealing tray 24 having a plurality of recesses 42 for receiving the second ends of the plurality of capsular devices 8. The sealing tray 24 can be provided with a squared-off corner 25. Each recess 42 has a sidewall 44 and a bottom wall 46, and is designed to seal the second end opening 18 of a respective capsular device 8. An optical adhesive can be provided in each recess, and the optical adhesive can be optically cured after preliminary construction of the assembly. The sealing tray 24 can be designed to have the same length and width dimensions as the array tray 22, the same number of recesses 42 as through-holes 32 in the array tray 22, and the same relative center-to-center spacing of recesses as the center-to-center spacing of the through-holes 32 of the array tray 22. The system 40 can initially be provided with the sealing tray 24, thus eliminating the need for sealing caps such as caps 16 shown in FIGS. 1 and 3.

FIG. 6 is a perspective view of the system 40 shown in FIG. 5 in an assembled state. The second ends 21 of the plurality of capsular devices 8 are received in respective recesses of the sealing tray 24. Optionally, one or more agents, reagents, or other components can be added prior to sealing. The second end openings 18 of the plurality of capsular devices 8 are sealed by the sealing tray 24. The sealed system can be archived for extended periods of time or subject to PCR, transcription, RT, RTPCR, or another process.

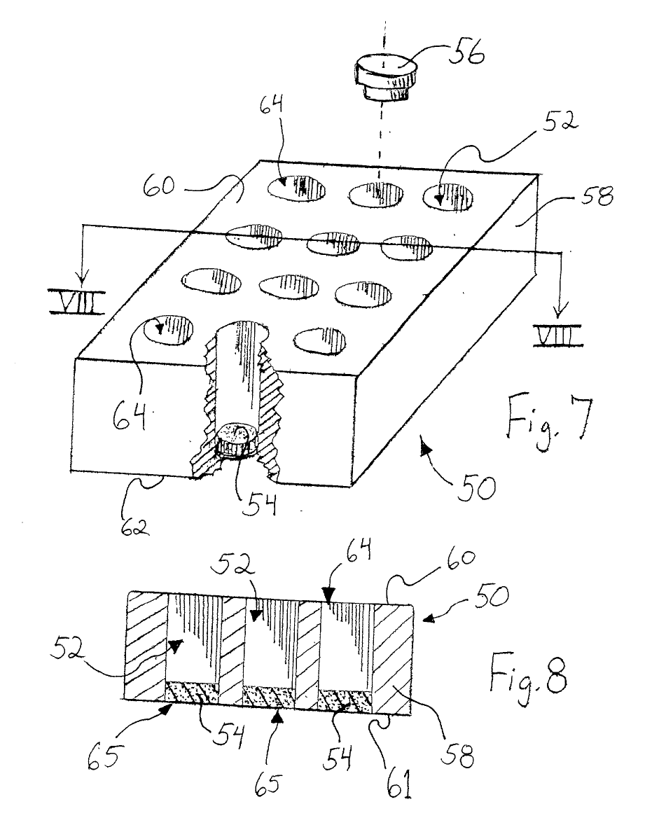

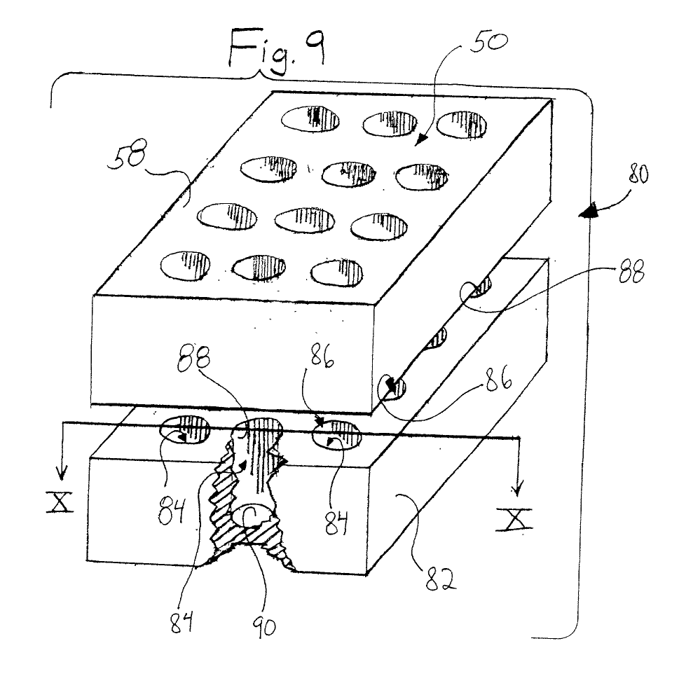

FIG. 7 is a perspective, partial cut-away view of yet another embodiment of the present invention wherein a plate system 50 includes a plate 58 having a top surface 60 and a bottom surface 62. FIG. 8 is a cross-sectional view taken through line VIII-VIII of FIG. 7. The plate 58 is provided with a plurality of through-holes 52 and a species-immobilizing filter 54 disposed in each through-hole 52. In the embodiment shown, first openings 64 of the through-holes 52 are located at the top surface 60 of the plate 58. The first openings 64 can be sealed with respective removable end caps 56, one end cap 56 being shown, or with any other sealing device (not shown) such as an adhesive sheet, a sealing tray, or a sealing member. Likewise, second or bottom, end openings 65 can be sealed with removable caps or with any other suitable sealing device.

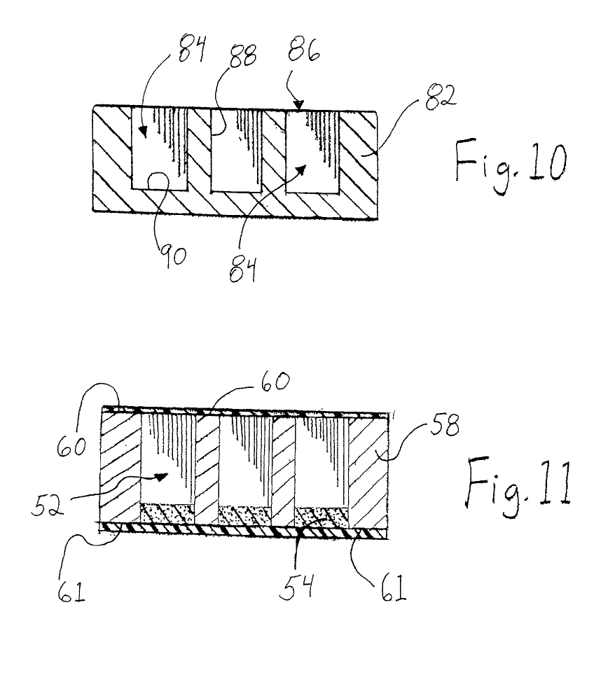

FIG. 9 is a perspective exploded view of an assembly 80 including the plate system 50 shown in FIG. 7, disposed above a waste collection tray 82. FIG. 10 is a cross-sectional view of the waste collection tray 82 shown in FIG. 9 taken through line X-X of FIG. 9. The waste collection tray 82 is provided with waste collection wells 84. Each well 84 has an opening 86, a sidewall 88, and a bottom wall 90. The diameter of the opening 86 is the same as the diameter of the second end opening 65 of the plate 58. The waste collection tray 82 collects waste washed-through or passed-through the through-holes 52 of the system 50. Registering means can be provided to align the plate system 50 with the waste collection tray 82.

FIG. 11 is a cross sectional view of the plate system 50 shown in FIG. 7 having as sealing devices a sealing top plate 90 and a sealing bottom plate 92 adhered to the respective top surface 60 and bottom surface 61 of the plate system 58.

In the embodiment of a device of the present invention as illustrated in FIGS. 7-11, a removable protective lid (not shown), initially present on the plate 58, can be removed at the beginning of an operation. Biological samples, for example, such as whole blood, cells, and/or tissue extracts, can be pipetted manually or robotically into the through-holes of the plate 58. The samples can be adsorbed onto or absorbed into the filter 54. The through-holes 52 can then be sealed with or without additional components, and archived for an extended period of time. The samples can be allowed to dry. Alternatively, or additionally, a purification or wash solution can be made to wash the filter and purify and/or isolate a target analyte on or in the filter. Washing can be facilitated with the use of a vacuum, for example, either by manual placement or robotic placement in a vacuum flow. Archiving can occur before or after purification. The purified sample can then be sealed in the plate, with or without additional components, and archived or subjected to PCR, transcription, RT, RTPCR, or another process.

The plate 58, after sample purification, can be sealed and archived, and/or transferred by either manual or robotic means to a platform or a workstation. Additional components such as PCR solution, transcription solution, RT solution, or RTPCR solution can be added to the through-holes 52 before sealing. The plate 58 can be sealed with an adhesive or heat-sealable membrane material, for example, to create a permanent bond to either the top surface 60, bottom surface 61, or both surfaces 60, 61 of the plate 58. As such, leak-proof seals can be provided and the device can be converted to a PCR plate, a transcription plate, an RT plate, an RTPCR plate, or a similar device.

The present invention also relates to methods of using the capsule and tray assemblies described herein in automated laboratory workstations to perform sequential chemical reactions, such as collections, purifications, isolations, PCR, transcription, RT, or RTPCR. Thus, the invention further provides a method for separating at least one target analyte comprising a nucleic acid or nucleic acid fragment, wherein the method includes providing a nucleic acid or nucleic acid fragment sample purification device including a tubular body having a first end, a first end opening, a second end, a second end opening, and a species-immobilizing filter within the tubular body, where the species-immobilizing filter is capable of isolating a target analyte and immobilizing the analyte on or in the filter. The method further includes introducing a biological sample including a nucleic acid or nucleic acid fragment through an end opening of the tubular body and causing target analyte, if present in said sample, to be immobilized by the species-immobilizing filter. Components of the biological sample other than the target analyte are then removed from the species-immobilizing filter. Alternatively, target analyte can be immobilized on or in the filter through any other filtering mechanism. In this manner, the present invention provides a testing method and a use of the method. Thus, a patient's blood sample or a component thereof may, by a method of the present invention, be placed in the purification device, retained by the species-immobilizing filter, and subjected to reaction conditions whereby a reaction product results from the sample. The product of the reaction can be analyzed for the presence of a particular product and a conclusion can be drawn regarding the patient's blood.

The invention further provides a method for performing a nucleotide polymerization reaction or other nucleic acid or nucleic acid fragment manipulation reaction, including an amplification and detection reaction such as used in the INVADER technology available from Third Wave Technologies of Madison, Wisc., a PCR amplication of an oligonucleotide or nucleic acid fragment, transcription, RT, or RTPCR. The method includes providing a biological sample purification device, where the device includes a tubular body having a first end, a first end opening, a second end, a second end opening, and a species-immobilizing filter within the tubular body. A biological sample containing a target analyte is introduced into the tubular body through at least one of the first end opening and the second end opening of the tubular body. A reaction solution, for example, a polymerase chain reaction solution is introduced into the tubular body. The end openings are then sealed to form a sealed device, and the sealed device is exposed to conditions to affect a desired reaction, such as polymerase chain reaction amplification of the target analyte.

The invention further relates to a method of manipulating a target analyte from a biological sample. The method includes providing a biological sample purification device containing a tubular body having a first end, a first end opening, a second end, a second end opening, at least one removable cap attachable to at least one of the first and second ends, and a species-immobilizing filter within the tubular body. A biological sample containing a target analyte is introduced into the device and the target analyte from the sample is immobilized by the species-immobilizing filter. The target analyte is then isolated by removing other components of the biological sample from the species-immobilizing filter. The end opening(s) of the tubular body through which the biological sample is introduced is then sealed, such that both end openings are sealed with sealing devices, thereby forming a sealed device. The sealed device is then subjected to conditions that cause a desired reaction, for example, polymerase chain reaction amplification, of the target analyte within the device.

Other embodiments of the present invention will be apparent to those skilled in the art from consideration of the specification and practice of the invention disclosed herein. It is intended that the specification and examples be considered as exemplary only, with the true scope and spirit of the invention being indicated by the following claims.

What is claimed

1. A system for processing a plurality of fluid samples, said system comprising:
a plurality of biological sample purification devices, each device of said plurality of devices comprising a tubular body having a first end, a first end opening, a second end, a second end opening, a species-immobilizing filter held within the tubular body, and a removable cap adapted to seal the second end opening; and
a sealing device having a surface adapted to individually seal each of the first end openings of said plurality of devices.

2. The system of claim 1, wherein said surface has a plurality of recesses therein, said sealing device is adapted to receive the first ends of said plurality of devices in respective ones of said recesses, and said sealing device is adapted to seal the first end openings of said plurality of devices when the respective first ends of said plurality of devices are received in said recesses.

3. The system of claim 1, wherein each device of said plurality of devices further includes a second removable cap adapted to seal the first end opening of the respective device.

4. The system of claim 1, wherein at least one device of said plurality of devices has the respective removable cap attached to the second end of the device and the respective second removable cap attached to the first end of the device.

5. The system of claim 3, wherein each device of said plurality of devices includes the respective removable cap attached to the second end of the device and the respective second removable cap attached to the first end of the device.

6. The system of claim 1, wherein said species-immobilizing filter of each device is positioned within the tubular body of the respective device such that the ratio of (1) the distance from the filter to the first end, to (2) the distance from the filter to the second end, is greater than or equal to about 4:1.

7. The system of claim 1, wherein said species-immobilizing filter of each device is positioned at the second end of the respective tubular body.

8. The system of claim 1, wherein the first end opening of each device of said plurality of devices is sealed with said sealing device, and said sealing device includes an adhesive.

9. The system of claim 8, wherein the adhesive is optically-curable, pressure sensitive, or both.

10. The system of claim 1, wherein said species-immobilizing filter of each device comprises a nucleic acid purification membrane.

11. The system of claim 1, further including a target analyte bound to the species-immobilizing filter of at least one device of said plurality of devices, said target analyte comprising a nucleic acid or nucleic acid fragment.

12. The system of claim 11, wherein said at least one device that includes said target analyte also contains a polymerase chain reaction solution, a transcription solution, a reverse transcription solution, or a reverse transcription polymerase chain reaction solution.

13. The system of claim 1, further including a biological sample that comprises an animal cell lysate or a plant cell lysate, within the tubular body of at least one device of said plurality of devices.

14. The system of claim 1, further including a biological sample that comprises whole blood, within the tubular body of at least one device of said plurality of devices.

15. The system of claim 1, further including a biological sample that comprises tissue extract, within the tubular body of at least one device of said plurality of devices.

16. A purification apparatus including the system of claim 1, wherein the first end opening of each device of said plurality of devices is sealed by said sealing device in the form of an assembly.

17. The purification apparatus of claim 16, wherein said assembly further comprises a second sealing device, said second sealing device having a surface adapted to seal the second end openings of said plurality of devices.

18. The purification apparatus of claim 17, wherein said surface of said second sealing device has a plurality of recesses therein, said second sealing device is adapted to receive the second ends of two or more of said devices in respective ones of said recesses, and said second sealing device is adapted to seal the second end openings of said plurality of devices when the respective second ends of said devices are received in said recesses.

19. A purification apparatus including the system of claim 2, wherein each device of said plurality of devices is positioned with the respective first end thereof received within a corresponding one of said recesses in said sealing device, in the form of an assembly.

20. A method for manipulating at least one biological sample, said method comprising:
providing a biological sample purification device comprising a tubular body having a first end, a first end opening, a second end, and a second end opening, and a species-immobilizing filter within the tubular body;
introducing a biological sample into the tubular body through at least one of the first end opening and the second end opening of the tubular body;
causing a pressure differential across said species-immobilizing filter to immobilize a target analyte, if present in said sample, on or in said filter;
after said causing a pressure differential, sealing at least one of said first and second end openings with a sealing device, said sealing device having a surface adapted to seal at least one of the first and second end openings of a plurality of said biological sample purification devices, to form a sealed device; and
subsequently analyzing said device to determine the presence or absence of the target analyte or a reaction product thereof, in said device.

21. The method of claim 20, wherein said method further includes sealing at least one of said first and second end openings with a removable cap.

22. The method of claim 20, wherein a plurality of said devices is provided, and said method comprises:
introducing biological samples into the respective tubular body of each device of said plurality of devices through at least one of the respective first and second end openings of the respective tubular body;
causing a pressure differential across said species-immobilizing filter of each device to immobilize a target analyte, if present in the respective biological sample, on or in said filter of each device;
after said causing a pressure differential, sealing at least one of said first and second end openings of each device with said sealing device, to form said sealed device; and
subsequently analyzing each of said devices to determine the presence or absence of the target analyte or a reaction product thereof, in each said device.

23. The method of claim 20, wherein the surface of said sealing device includes an adhesive and said method includes adhering said sealing device and at least one of said first and second ends of said tubular body together.

24. The method of claim 23, wherein the adhesive is ultraviolet radiation-curable and said method includes curing said adhesive with ultraviolet radiation.

25. The method of claim 20, wherein said species-immobilizing filter comprises a nucleic acid purification membrane.

26. The method of claim 20, wherein said biological sample includes a target analyte and said method includes binding said target analyte to said species-immobilizing filter, said target analyte comprising a nucleic acid or nucleic acid fragment.

27. The method of claim 26, wherein said device contains a polymerase chain reaction solution, a transcription solution, a reverse transcription solution, or a reverse transcription polymerase chain reaction solution.

28. The method of claim 20, wherein said biological sample comprises an animal cell lysate, a plant cell lysate, whole blood, or a tissue extract.

29. The method of claim 20, further comprising introducing a reaction solution into the tubular body after said causing a pressure differential and before said sealing.

30. The method of claim 29, further comprising exposing the biological sample and reaction solution in said tubular body to conditions to effect a reaction.

31. The method of claim 20, wherein the species-immobilizing filter comprises a receptor capable of binding to a target analyte.

32. The method claim 20, further comprising archiving said device, after introducing the biological sample, for at least about 100 hours before said analyzing.

33. A method for archiving at least one biological sample, said method comprising:
providing a biological sample purification device, said device comprising
a tubular body having a first end, a first end opening, a second end, and a second end opening,
a species-immobilizing filter within the tubular body between said first end opening and said second end opening;
introducing a biological sample into the tubular body through at least one of the first end opening and the second end opening;
sealing at least one of said first and second end openings with a sealing device, such that both end openings are sealed to form a sealed device, said sealing device having a surface adapted to seal at least one respective end opening of each of a plurality of said devices; and
archiving the sealed device.

34. The method of claim 33, wherein said surface of said sealing device has a plurality of recesses, each recess adapted for receiving at least one of said first and second ends and adapted to seal at least one of said first and second end openings.

35. The method of claim 33, wherein said device includes at least one removable cap attached to at least one of the first and second ends.

36. The method of claim 33, wherein the surface of said sealing device includes an adhesive and said method includes adhering said sealing device and at least one of said first and second ends of said tubular body together.

37. The method of claim 36, wherein the adhesive is ultraviolet radiation-curable and said method includes curing said adhesive with ultraviolet radiation.

38. The method of claim 33, wherein said species-immobilizing filter comprises a nucleic acid purification membrane.

39. The method of claim 33, wherein said biological sample includes a target analyte and said method includes binding said target analyte to said species-immobilizing filter, said target analyte comprising a nucleic acid or nucleic acid fragment.

40. The method of claim 33, wherein said biological sample comprises an animal cell lysate, a plant cell lysate, whole blood, or tissue extract.

41. The method of claim 33, further comprising introducing a reaction solution into the tubular body before said sealing.

42. A method for separating at least one target analyte comprising a nucleic acid or nucleic acid fragment, from a biological sample, said method comprising:
providing a nucleic acid or nucleic acid fragment sample purification device, said device comprising a tubular body having a first end, a first end opening, a second end, a second end opening, and a species-immobilizing filter within the tubular body;
introducing a biological sample including a nucleic acid or nucleic acid fragment through an end opening of the tubular body;
causing target analyte, if present in said sample, to be immobilized on or in said species-immobilizing filter;
removing components of said biological sample other than said target analyte, from said species-immobilizing filter; and
sealing at least one of said first and second end openings with a sealing device, such that both end openings are sealed to form a sealed device, said sealing device having a surface adapted to seal at least one respective end opening of each of a plurality of said purification devices.

43. The method of claim 42, wherein said causing target analyte to bind to the species-immobilizing filter includes lysing whole blood cells containing said target analyte to release target analyte from said whole blood cells.

44. The method of claim 42, wherein said whole blood cells are lysed after said biological sample is introduced into said tubular body.

45. The method of claim 42, wherein a lysing agent is introduced into the tubular body before said biological sample is introduced into the tubular body, said lysing agent lysing components of said biological sample.

46. The method of claim 42, wherein a lysing agent is introduced into the tubular body after said biological sample is introduced into the tubular body, said lysing agent lysing components of said biological sample.

47. The method of claim 42, further comprising introducing a reaction solution into the tubular body before said sealing, and after sealing exposing the biological sample and a reaction solution in said tubular body to conditions to effect a reaction.

48. An analytical system for manipulating biological samples, comprising;
a plate having a first surface and a second surface that opposes said first surface, and a plurality of through-holes, each through-hole extending from said first surface to said second surface and defining a first end opening at said first surface and a second end opening at said second surface;
a plurality of species-immobilizing filters, each disposed within a respective one of said through-holes; and
a first sealing device adapted to individually seal each first end opening of said plurality of through-holes; and
a second sealing device adapted to seal each second end opening of said plurality of through-holes.

49. The system of claim 48, wherein said first sealing device comprises a plurality of removeable end caps adapted to individually seal the first end openings of said plurality of through-holes.

50. The system of claim 48, wherein at least one of said first sealing device and said second sealing device includes a sealing tray having recesses adapted to individually seal: the first end openings; the second end openings; or both the first end openings and the second end openings, of said plurality of through-holes.

51. A method of manipulating a biological sample comprising:
collecting a biological sample in or on one or more of the plurality of species-immobilizing filters of the system of claim 48.

52. The method of claim 51, further comprising purifying a target analyte by retaining said target analyte on or in said species-immobilizing filter, and separating said target analyte from other components in a sample introduced in the through-holes of said system.

53. A method comprising: introducing a biological sample including a target analyte in a first through-hole of the system of claim 48, such that said target analyte is immobilized in or on the respective species-immobilizing filter within the first through-hole;
introducing a solution into at least said first through-hole, said solution having sufficient components to enable a reaction of the target analyte; and
subsequent to introducing said sample and said solution, sealing the first end openings and second end openings of said system to form a sealed system.

54. The method of claim 53, wherein said solution comprises a polymerase chain reaction solution, a transcription solution, a reverse transcription solution, and a reverse transcription polymerase chain reaction solution, or a combination thereof.

55. The method of claim 54, wherein said solution is a polymerase chain reaction solution and said method further comprises subjecting the sealed system to conditions for affecting polymerase chain reaction of the target analyte.

56. A method of archiving a biological sample, comprising:
Introducing a biological sample into one or more through-holes of the plurality of through-holes of the system of claim 48;
sealing the first end openings and the second end openings of said system to form a sealed device; and
archiving said sealed device.

57. The method of claim 56, wherein said sealed device is archived for a period of time of about one hundred (100) hours or greater.
